(19) United States
(12) Patent Application Publication
(10) Pub. No.: US 2009/0005762 A1
(43) Pub. Date: Jan. 1, 2009

(54) Bioabsorbable Drain Tube

(76) Inventors: Vasu Nishtala, Snellville, GA (US); Michael O'Grady, Monroe, GA (US); Robert Orr, Covington, GA (US); Robert Young, Loganville, GA (US)

Correspondence Address: BRIAN M. BURN, ESQ.;C. R. BARD MEDICAL DIVISION, P.O. BOX 52050, c/o PORTFOLIOIP, MINNEAPOLIS, MN, 55402, US

(21) Appl. No.: 11/768,876

(22) Filed: Jun. 26, 2007

(51) Int. Cl. A61M 27/00

(52) U.S. Cl. 604/541

(57) ABSTRACT

A bioabsorbable drain tube for the evacuation of fluids and gases from a body cavity or wound to promote healing, such as after chest surgery. The bioabsorbable drain tube includes a first elongated section molded from a bioabsorbable polymer, the first elongated section having a first end, a second end and a plurality of drain openings; and a second elongated section, the second elongated section having a first end and a second end, the first end of the second elongated section in fluid communication with the second end of the first elongated section. A method of draining fluid from a surgical site or wound of a patient is also provided.

### FIELD OF THE INVENTION

This invention relates to the field of medical devices and, more particularly, to improved surgical drain tubes.

### BACKGROUND OF THE INVENTION

In the evacuation of fluids and gases from a body cavity or wound to promote healing, such as after chest surgery, a drain tube is often employed to drain the fluid from the cavity into a collection system. The drainage site may be a natural body cavity or an orifice or may be surgically formed. Drainage is generally facilitated by gravity or with the assistance of suction.

Devices that drain surgical incisions typically include an implantable, inflow surgical drain that is, at least partially, placed within the patient's body where it is in communication with bodily materials. This drain is usually connected to an elongated transitional tube that leads from the inflow section and extends towards the outside of the patient's body. This, in turn, is connected to an outflow section, which is connected to a vacuum device and a suitable reservoir for receiving the bodily materials collected from the drain.

Drainage systems are closed if they capture the fluids with tubing coupled to a closed container or canister and are open if fluid is accumulated in gauze or corrugated rubber sheeting. Canisters and collapsible vessels use vacuum or the restoration force of the collapsed vessel to provide respectively, active high or low drainage pressure. Completely passive drains operate on the pressure differential between the inside and outside of the body.

Typically, drains available for surgical incisions have either a series of small, parallel perforations that run the length of the drain, or a series of narrow, linear channels. Unwanted bodily materials are drawn into the drain through the perforations or channels and drawn through the transition component, the outflow component and into the reservoir. The drains are usually of a flattened oval or a substantially circular shape and are usually about 30 cm long.

The channel drain has proven to lower the risk of occlusion and premature drain removal, minimizes tissue invagination and reduces the incidence of seroma formation. Further, channel drains typically possess an increased drainage area, due to their multiple channels, permitting superior drainage. An additional benefit stems from the fact that the channel drain has no weak points that would cause it to tear during normal usage, since it typically has three or four longitudinal grooves into which the body fluids enter.

There are several fairly common problems with current surgical drains. In drains that have a series of small parallel perforations, such as a drain known as the Jackson-Pratt drain, the perforations can act as weak points in the structure of the drain and can break or tear when the drain is pulled during extraction from the wound. If this occurs, an incision is required to remove the drain.

Another deficiency with perforated drains is their length. Most current drains were not designed for the long tunnels in wounds created in an appreciable number of the current minimally-invasive surgeries. The most convenient way to overcome this deficiency at present is to implant two or more drains into a wound. While easy to do, it requires more than one drain to be extracted, thereby increasing the patient's discomfort.

Another problem encountered with the using of existing drains stems from the fact that the vacuum applied to the drain tube often draws adjacent internal tissue into the drain resulting in restriction of flow, often requiring withdrawal and removal and replacement of the drain tube. Additional trauma to the wound or surgical opening and interference with the healing process are detrimental and undesired consequences associated with this problem. Moreover, any internal tissue drawn into the drain tube makes the removal of the drain tube more difficult and potentially much more painful for the patient.

As noted by Bruce E. A., Howard R. F. and Franck L. S. in Journal of Clinical Nursing, February, 2006; 1 Vol. 5(2):pages 145-154, the removal of a chest drain is a painful and frightening experience, particularly for children, as evidenced by existing research regarding the amount of pain experienced and effectiveness of analgesia with this procedure. The majority of studies indicate that patients experience moderate to severe pain during chest drain removal, even when morphine or local anesthetics were given. As such, it was concluded that morphine alone does not provide satisfactory analgesia for chest drain removal pain and that non-steroidal anti-inflammatory drugs, local anesthetics and inhalation agents may have a role to play in providing more effective analgesia for this procedure.

Despite the advances in the art, a need exists for an improved drainage tube system that provides the therapeutic effect of promoting drainage from wound cavities, without subjecting the patient to the pain normally associated with the drain removal process. Therefore, what is needed is a drain that does not require removal upon completion of the drainage process.

### SUMMARY OF THE INVENTION

In one aspect, provided is a bioabsorbable drain tube for the evacuation of fluids and gases from a body cavity or wound to promote heating, such as after chest surgery. The bioabsorbable drain tube includes a first elongated section molded from a bioabsorbable polymer, the first elongated section having a first end, a second end and a plurality of drain openings and a second elongated section, the second elongated section having a first end and a second end, the first end of the second elongated section in fluid communication with the second end of the first elongated section.

In another aspect, provided is a method of draining fluid from a surgical site or wound of a patient. The method includes the steps of inserting a bioabsorbable drain tube into the surgical site or wound of a patient, positioning the bioabsorbable drain tube within a region of fluid accumulation of the surgical site or wound, placing collection means in fluid communication with the bioabsorbable drain tube and accumulating fluid in the collection means.

The bioabsorbable drain tubes disclosed herein may be provided with any number of lumens, including one, two, three, four or more lumens, and may be of any cross-section, including substantially circular, oval or octagonal. The bioabsorbable drain tubes may also be of the perforated or channel type.

The channel-type bioabsorbable drain tubes disclosed herein include a first elongated section that includes an axial core having a longitudinal axis, the axial core having a plurality of radial ribs projecting radially along the longitudinal axis. The radial ribs can be of equal length and have a first end and a second end, each second end terminating at the axial core's periphery and spaced equally about the axial core. Each first end of each radial rib terminates in an outer peripheral member, the outer peripheral member extending longitudinally about the length of each radial rib. The outer peripheral members are sized to form a segmented circle or oval at the first elongated section's periphery, with gaps between adjacent outer peripheral members. Each gap forms a drain opening parallel to the longitudinal axis of the axial core and extends throughout the length of the first elongated section.

The bioabsorbable drain tubes may be produced from any of the known biocompatible, bioabsorbable polymers, including poly(lactide) poly(glycolide), poly(dioxanone), poly(ε-caprolactone), poly(hydroxybutyrate), poly(β-hydroxybutyrate), poly(hydroxyvalerate), poly(tetramethyl carbonate), poly(lactide-co-glycolide), poly(amino acids) and copolymers, terpolymers and blends thereof.

These and other features will be apparent from the detailed description taken with reference to accompanying drawings.

### BRIEF DESCRIPTION OF THE DRAWINGS

The invention is further explained in the description that follows with reference to the drawings illustrating, by way of non-limiting examples, various embodiments of the invention wherein:

FIG. 1 is a partial perspective view of a bioabsorbable drain tube of the present invention, depicting a three-lumen elongated drain section;

FIG. 2 is a cross-sectional view of the bioabsorbable drain tube of FIG. 1 taken along line 2-2 of FIG. 1;

FIG. 3 is a partial perspective view of a bioabsorbable drain tube of the present invention, depicting a four-lumen elongated drain section;

FIG. 4 is a cross-sectional view of the bioabsorbable drain tube of FIG. 3 taken along line 4-4 of FIG. 3;

FIG. 5 is a partial perspective view of a bioabsorbable drain tube of the present invention, depicting an offset, three-lumen elongated drain section;

FIG. 6 is a cross-sectional view of the bioabsorbable drain tube of FIG. 5 taken along line 6-6 of FIG. 5;

FIG. 7 is a partial perspective view of a bioabsorbable drain tube of the present invention, depicting a tubular, single-lumen elongated drain section of circular cross-section;

FIG. 8 is a cross-sectional view of the bioabsorbable drain tube of FIG. 7 taken along line 8-8 of FIG. 7;

FIG. 9 is a partial perspective view of a bioabsorbable drain tube of the present invention, depicting a tubular, single-lumen elongated drain section of octagonal cross-section;

FIG. 10 is a cross-sectional view of the bioabsorbable drain tube of FIG. 9 taken along line 10-10 of FIG. 9;

FIG. 11 is a partial perspective view of a second embodiment of the present invention showing a four-lumen oval or flat drain; and

FIG. 12 is a cross-sectional view of the drain of the second embodiment taken along lines 12-12 of FIG. 11.

### DETAILED DESCRIPTION OF PREFERRED EMBODIMENTS

Reference is now made to FIGS. 1-12, wherein like numerals are used to designate like parts throughout.

The bioabsorbable drain tubes disclosed herein find utility in the evacuation of fluids and gases from body cavities or wounds in order to promote healing, such as after chest surgeries. Polymers contemplated for use in the manufacture of the bioabsorbable drain tubes disclosed herein include the class of polymers known as bioabsorbable polymers. These biocompatible polymers include, but are not limited to, poly(lactide), including the L (−), D (+), meso and racemic lactide form, poly(glycolide), poly(dioxanone), poly(ε-caprolactone), poly(hydroxybutyrate), poly(β-hydroxybutyrate), poly(hydroxyvalerate), poly(tetramethyl carbonate), and poly(amino acids) and copolymers and terpolymers thereof. Also contemplated herein is a copolymer blend comprising poly(lactide)-co-poly(glycolide).

As indicated above, contemplated for use herein is polylactic acid (PLA or poly(lactide)), which is prepared from the cyclic diester of lactic acid (lactide) by ring opening polymerization, as shown below.

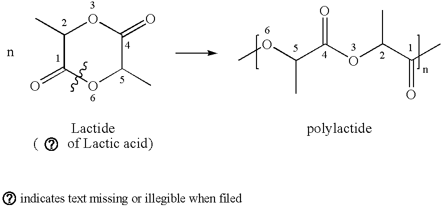

As may be appreciated, lactic acid exists as two optical isomers or enantiomers. The L-enantiomer occurs in nature, a D, L racemic mixture results from the synthetic preparation of lactic acid. Crystalline poly-L-lactide is more resistant to hydrolytic degradation than the amorphous DL form.

Unlike PLA, which is absorbed slowly, PGA is absorbed within a few months post-implantation, due to greater hydrolytic susceptibility. In vitro experiments have shown an effect on degradation by enzymes, buffer, pH, annealing treatments, and gamma irradiation. Acceleration of in vivo degradation due to gamma irradiation has been exploited to create devices where early fragmentation is desired.

Polyglycolic acid (PGA or poly(glycolide)) is a totally synthetic absorbable polymer contemplated for use herein and produced by the reaction shown below.

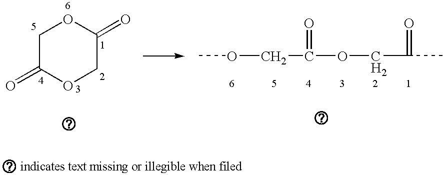

Also contemplated for use herein are copolymers of PGA and PLA, namely poly(lactide-o-glycolide). The copolymers are amorphous between the compositional range 25 to 70 mole percent glycolide. Pure polyglycolide is about 50% crystalline, whereas pure poly-L-lactide is reported to be about 37% crystalline. Like pure PGA and pure PLA, a 90/10 PGAPLA is also weakened by gamma irradiation. Another approach to copolymerization includes using a starting monomer that is neither lactide nor glycolide, but rather an unsymmetrical cyclic diester containing one lactate and one glycolate moiety. This monomer produces a polymer with the same empirical formula as poly(lactide-co-50%-glycolide), but possesses different properties due to a more stereoregular configuration.

Another polymer contemplated for use herein is polydioxanone. The monomer p-dioxanone, is analogous to glycolide but yields a poly-(ether-ester) as shown below.

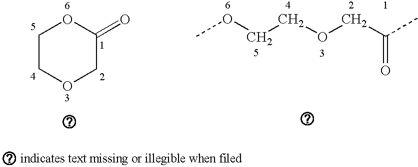

Poly(dioxanone) is known to retain tensile strength longer than polyglycolide and is absorbed within about six months with minimal tissue response. Poly(dioxanone) degradation in vitro is affected by gamma irradiation dosage, but not substantially by the presence of enzymes.

Also contemplated for use herein is the polymer poly(ε-caprolactone). Poly(ε-caprolactone) is synthesized from ε-caprolactone, as shown below.

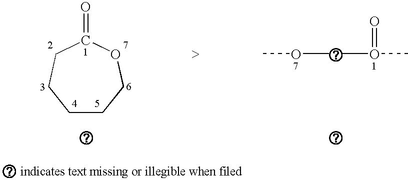

Additionally, copolymers of ε-caprolactone and L-lactide are contemplated for use herein. They are known to be elastomeric when prepared from 25% ε-caprolactone, 75% L-lactide and rigid when prepared from 10%-caprolactone, 90% L-lactide.

Also contemplated for use herein are the bioabsorbable polymers poly(hydroxybutyrate) and poly(hydroxyvalerate), shown below.

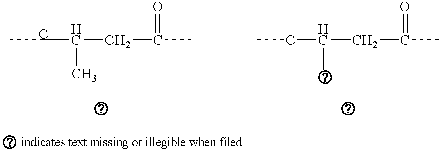

Poly(β-hydroxybutyrate) (PHB) is a biodegradable polymer that occurs both in nature and can easily be synthesized in vitro. Synthetic PHB, however, has not shown the stereoregularity found in the natural product. High MW, crystalline, and optically active PHB have been extracted from bacteria, PHB polymer is melt processable and has been proposed for use as an absorbable suture. Recent improvements in the extraction process have resulted in renewed interest in PHB for both medical and nonmedical applications. Copolymers of hydroxybutyrate and hydroxyvalerate have been developed to provide a wide variety of mechanical properties and more rapid degradation than can be achieved with pure PHB, and are also contemplated for use herein.

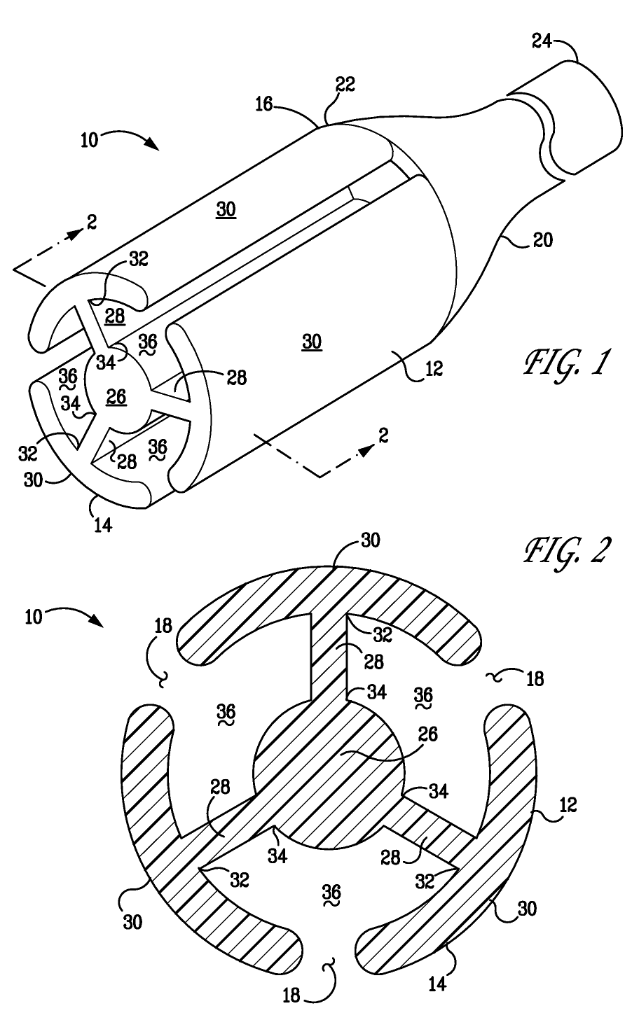

Referring now to FIGS. 1 and 2, one exemplary embodiment of a bioabsorbable drain tube 10 includes a first elongated section 12 molded from a bioabsorbable polymer of a type described hereinabove. First elongated section 12 has a first end 14, a second end 16 and a plurality of drain openings 18. Bioabsorbable drain tube 10 also includes a second elongated section 20 having a first end 22 and a second end 24. As shown, the first end 22 of the second elongated section 20 is in fluid communication with the second end 16 of first elongated section 12.

The first elongated section 12 and a small part of the second elongated section 20 are placed in a patients body with the first elongated section 12 in fluid communication with a wound. According to one embodiment, second elongated section 20 is connected to a sealed, sterilized suction device (not shown) for drawing fluid through the bioabsorbable drain tube 10. As may be appreciated by those skilled in the art, the second elongated section 20 can exit the patient's body through an aperture formed in healthy tissue, adjacent to the wound. Further, the second elongated section 20 can have a smooth exterior to permit the surface tissue surrounding the aperture to seal against the exterior of the second elongated section 20, and thus, prevent the passage of air. This permits the wound to be completely closed, as by sutures, and covered with a dressing to form an aseptic barrier, thereby sealing the wound from the atmosphere. Thus, since the first elongated section 12 is in contact only with the sterile suction device, and not the atmosphere, the risk of infection is reduced.

Still, referring to FIGS. 1 and 2, bioabsorbable first elongated section 12 is shown to be of a fluted configuration, which may be radially symmetrical, as shown. Other configurations will advantageously benefit from the novel features disclosed herein, as will be described in more detail below. As shown, first elongated section 12 includes an axial cylindrical core 26, with a plurality of radial ribs 28 projecting radially from the axial cylindrical core 26 along its longitudinal axis. The radial ribs 28 are of equal length and have a first end 32 and a second end 34, each second end 34 terminating at the periphery of the axial cylindrical core 26. The radial ribs 28 may be spaced equally about axial cylindrical core 26, as shown. Each first end of each radial rib 32 terminates in an outer peripheral member 30 and extends longitudinally about the length of each radial rib 28. Viewed cross-sectionally, as best seen in FIG. 2, each outer peripheral member 30 is a thin arcuate member, symmetrically positioned about its respective radial rib 32. The outer peripheral members 30 are sized to form a segmented circle at the periphery of the first elongated section 12, with small gaps between adjacent outer peripheral members 30. Each of these gaps forms a drain opening 18, parallel to the longitudinal axis of the axial cylindrical core 26, and extending throughout the length of the first elongated section 12, as shown in FIG. 1.

As shown, the axial cylindrical core 26, radial ribs 28, drain openings 18 and outer peripheral members 30 cooperate to form plural channels or lumens 36 along the length of first elongated section 12. The lumens 36 permit fluid communication between one of the lumens 36 and the wound. The width of the lumens 36 may be about 0.05 to 0.2 times the outside diameter of the first elongated section 12. This configuration serves to provide adequate tissue contact drainage area while inhibiting tissue growth or entry of debris, such as clots, into the lumens 36.

The second end of the first elongated section 12 and the first end of the second elongated section 20 may be connected together in abutting relationship by means of a flexible tubular collar (not shown). As may be appreciated, such a collar serves to span the butt joint formed by the second end 16 of first elongated section 12 and the first end 22 of second elongated section 20 and may be affixed to first elongated section 12 and second elongated section 20 by a suitable adhesive material.

Second elongated suction section 20 may also be molded from a bioabsorbable polymer of the type described hereinabove or, alternatively, may be molded from any of a number of other suitable materials, such as a biocompatible elastomer like silicone. The use of silicone can serve to contribute to making the overall bioabsorbable drain tube soft and pliable, reducing patient discomfort and irritation of the wound, while still providing sufficient rigidity.

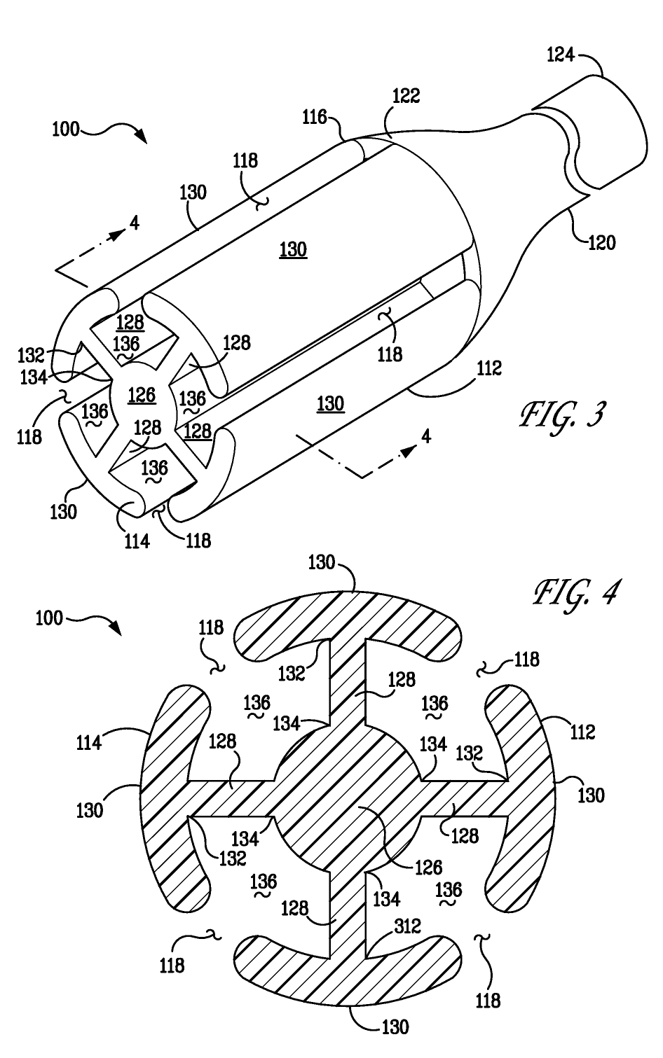

Although the embodiment of FIGS. 1 and 2 is shown as having three lumens 36, it will be recognized by those skilled in the art that other designs having a different number of lumens may also be provided. Referring now to FIGS. 3 and 4, a four-lumen design is shown. This exemplary embodiment of a bioabsorbable drain tube 100 includes a first elongated section 112, once again, molded from a bioabsorbable polymer of a type described hereinabove. First elongated section 112 has a first end 114 a second end 116 and a plurality of drain openings 118. Bioabsorbable drain tube 100 also includes a second elongated section 120 having a first end 122 and a second end 124, the first end 122 of the second elongated section 120 being in fluid communication with the second end 116 of first elongated section 112. According to one embodiment, second elongated section 120 is connected to a sealed, sterilized suction device (not shown) for drawing fluid through the bioabsorbable drain tube 100.

Bioabsorbable drain tube 100 may be radially symmetrical. As shown, first elongated section 112 includes an axial cylindrical core 126 with a plurality of radial ribs 128 projecting radially from the axial cylindrical core 126 along its longitudinal axis. The radial ribs 128 are ordinarily of equal length and have a first end 132 and a second end 134, each second end 134 terminating at the periphery of the axial cylindrical core 126. The radial ribs 128 may be spaced equally about axial cylindrical core 126, as shown.

Each first end of each radial rib 132 terminates in an outer peripheral member 130 and extends longitudinally about the length of each radial rib 128. As shown in FIG. 4, each outer peripheral member 130 is a thin arcuate member, symmetrically positioned about its respective radial rib 132. The outer peripheral members 130 are sized to form a segmented circle at the periphery of the first elongated section 112, with small gaps between adjacent outer peripheral members 130. Each of these gaps forms a drain opening 118, parallel to the longitudinal axis of the axial cylindrical core 126, and extending throughout the length of the first elongated section 112 as shown in FIG. 3.

As shown, the axial cylindrical core 126, radial ribs 128, and outer peripheral members 130 cooperate to form four plural channels or lumens 136 along the length of first elongated section 112. The width of the lumens 136 may be about 0.05 to 0.2 times the outside diameter of the first elongated section 112. Once again, this configuration serves to provide adequate tissue contact drainage area while inhibiting tissue growth or entry of debris, such as clots, into the lumens 136.

The second end of the first elongated section 112 and the first end of the second elongated section 120 may be connected together in abutting relationship by means of a flexible tubular collar (not shown). As may be appreciated, such a collar serves to span the butt joint formed by the second end 116 of first elongated section 112 and the first end 122 of second elongated section 120 and may be affixed to first elongated section 112 and second elongated section 120 by a suitable adhesive material.

Second elongated section 120 may also be molded from a bioabsorbable polymer of the type described hereinabove or, alternatively, may be molded from any of a number of other suitable materials, such as a biocompatible elastomer like silicone. Once again, the use of silicone can serve to contribute to making the overall bioabsorbable drain tube soft and pliable, reducing patient discomfort and irritation of the wound, while still providing sufficient rigidity.

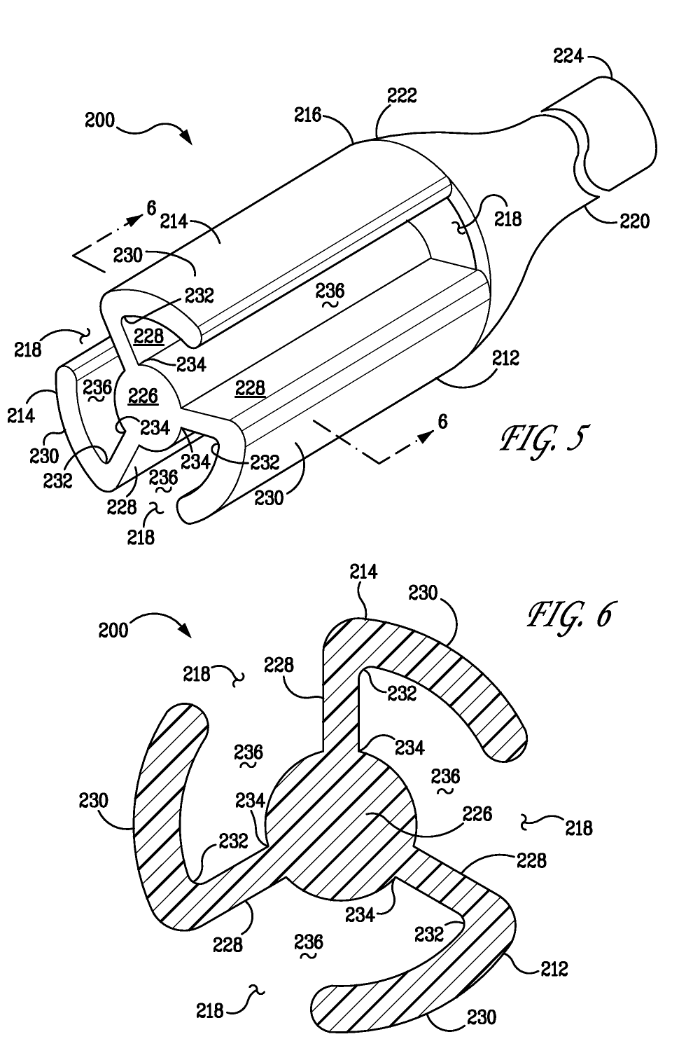

An offset variation of the three-lumen embodiment of FIGS. 1 and 2 is shown in FIGS. 5 and 6. Referring now to FIGS. 5 and 6, a bioabsorbable drain tube 200 includes a first elongated section 212, once again, molded from a bioabsorbable polymer of a type previously described, First elongated section 212 has a first end 214, a second end 216 and a plurality of drain openings 218. Bioabsorbable drain tube 200 also includes a second elongated section 220 having a first end 222 and a second end 224, the first end 222 of the second elongated section 220 in fluid communication with the second end 216 of first elongated section 212. According to one embodiment, second elongated section 220 is connected to a sealed, sterilized suction device (not shown) for drawing fluid through the bioabsorbable drain tube 200.

Bioabsorbable drain tube 200 is shown to be a three lumen configuration, which has an offset configuration, rather than being radially symmetrical, as in FIGS. 1 and 2. As shown in FIGS. 5 and 6, first elongated section 212 includes an axial cylindrical core 226, with a plurality of radial ribs 228 projecting radially from the axial cylindrical core 226 along its longitudinal axis. The radial ribs 228 are of equal length and have a first end 232 and a second end 234, each second end 234 terminating at the periphery of the axial cylindrical core 226. The radial ribs 228 may be spaced equally about axial cylindrical core 226, as shown.

Each first end of each radial rib 232 terminates in an outer peripheral member 230 and extends longitudinally about the length of each radial rib 228. As shown in FIG. 6, each outer peripheral member 230 is a thin arcuate member, positioned in an offset manner about radial rib 232. The outer peripheral members 230 are sized to form a segmented circle at the periphery of the first elongated section 212, with small gaps between adjacent outer peripheral members 230. Each of these gaps forms a drain opening 218, parallel to the longitudinal axis of the axial cylindrical core 226, and extending throughout the length of the first elongated section 212, as shown in FIG. 5.

As shown, the axial cylindrical core 226, radial ribs 228, and outer peripheral members 230 cooperate to form three channels or lumens 236 along the length of first elongated section 212. As indicated above, the width of the lumens 236 may be about 0.05 to 0.2 times the outside diameter of the first elongated section 212. This configuration again serves to provide adequate tissue contact drainage area while inhibiting tissue growth or entry of debris, such as clots, into the lumens 236.

As with the other embodiments described above, the second end of the first elongated section 212 and the first end of the second elongated section 220 may be connected together in abutting relationship by means of a flexible tubular collar (not shown). As may be appreciated, such a collar serves to span the butt joint formed by the second end 216 of first elongated section 222 and the first end 222 of second elongated section 220 and may be affixed to first elongated section 212 and second elongated section 220 by a suitable adhesive material.

Second elongated section 220 may also be molded from a bioabsorbable polymer of the type described hereinabove or, alternatively, may be molded from any of a number of other suitable materials, such as a biocompatible elastomer like silicone.

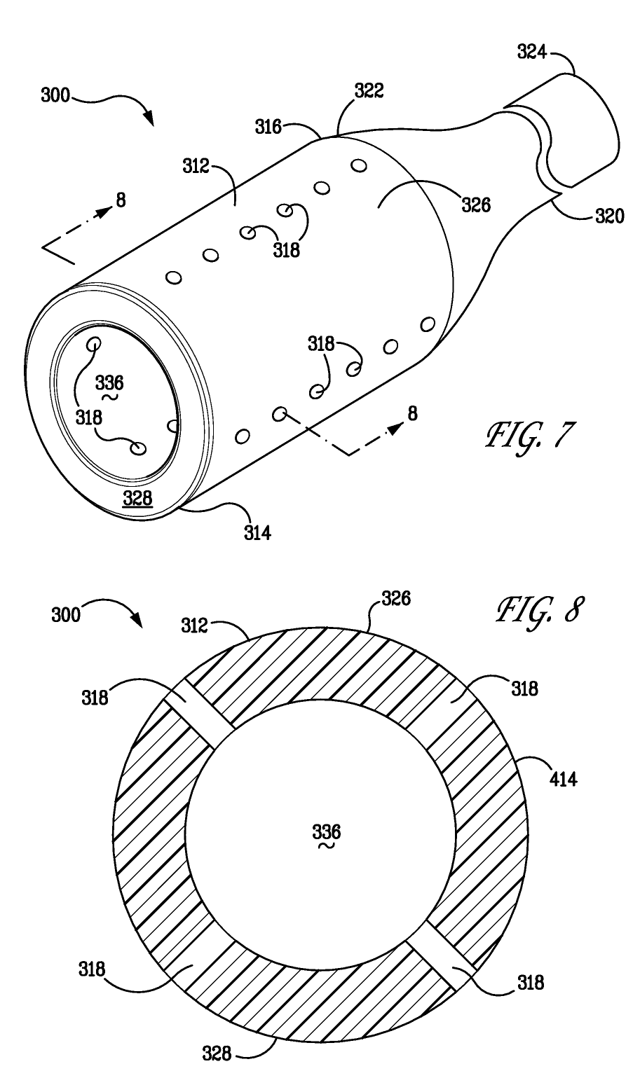

Referring now to FIGS. 7 and 8, another exemplary embodiment of a bioabsorbable drain tube 300 includes a first elongated section 312 molded from a bioabsorbable polymer of a type described hereinabove. First elongated section 312 has a first end 314, a second end 316 and a plurality of drain openings 318. Bioabsorbable drain tube 300 also includes a second elongated section 320 having a first end 322 and a second end 324. As shown, the first end 322 of the second elongated section 320 is in fluid communication with the second end 316 of first elongated section 312.

As with the channel-type designs of FIGS. 1-6, the first elongated section 312 and a small part of the second elongated section 320 are placed in a patient's body with the first elongated section 312 in fluid communication with a wound. Once again, second elongated section 320 may be connected to a sealed, sterilized suction device (not shown) for drawing fluid through the bioabsorbable drain tube 300.

Still referring to FIGS. 7 and 8, bioabsorbable drain tube 300 is shown to be of a cylindrical, tubular configuration. Other configurations will advantageously benefit from the novel features disclosed herein, as will be described in more detail below. As shown, first elongated section 312 includes a single lumen tube 326, with a plurality of drain openings 318 extending through the wall 328 of the single lumen tube 326, as shown in FIG. 3A. The single lumen 336 permits fluid communication with the wound. The number and diameter of drain openings 318 may be varied in accordance with the intended application so as to provide adequate tissue contact and drainage, while inhibiting tissue growth or entry of debris, such as clots, into the lumen 336.

The second end of the first elongated section 312 and the first end of the second elongated section 320 may be connected together in abutting relationship by means of a flexible tubular collar (not shown). As may be appreciated, such a collar serves to span the butt joint formed by the second end 316 of first elongated section 312 and the first end 322 of second elongated section 320 and may be affixed to first elongated section 312 and second elongated section 320 by a suitable adhesive material.

Second elongated suction section 320 may also be molded from a bioabsorbable polymer of the type described hereinabove or, alternatively, may be molded from any of a number of other suitable materials, such as a biocompatible elastomer like silicone.

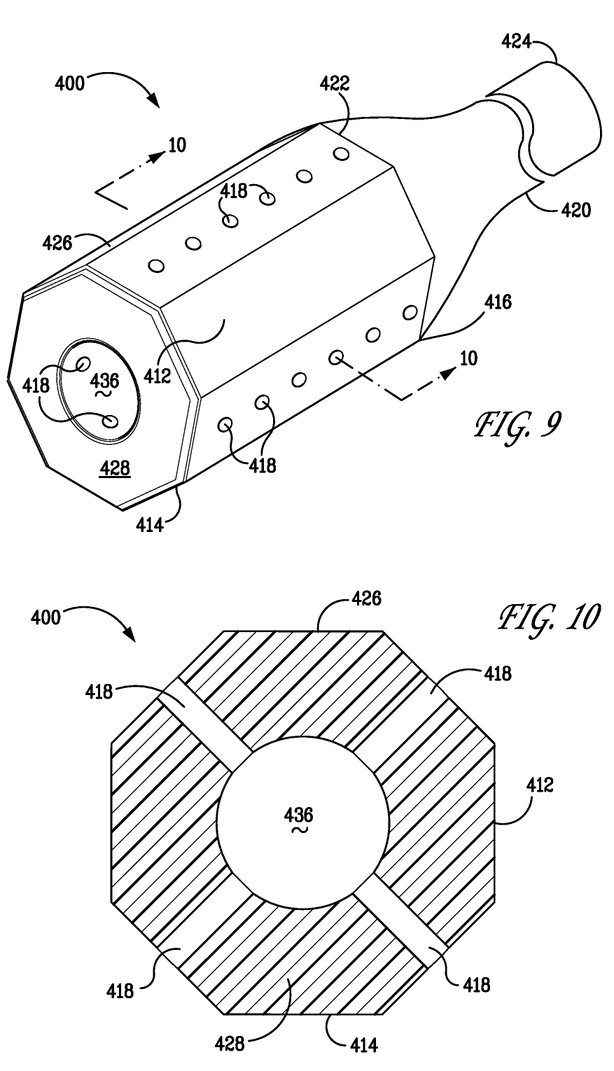

Referring now to FIGS. 9 and 10, another exemplary embodiment of a bioabsorbable drain tube 400 is shown, which includes a first elongated section 412 molded from a bioabsorbable polymer of a type described above. First elongated drain section 412 has a first end 414, a second end 416 and a plurality of drain openings 418. Bioabsorbable drain tube 400 also includes a second elongated section 420 having a first end 422 and a second end 424. As shown, the first end 422 of the second elongated section 420 is in fluid communication with the second end 416 of first elongated section 412.

As with the embodiment of FIGS. 7 and 8, the first elongated section 412 and a small part of the second elongated section 320 are designed to be placed in a patient's body with the first elongated section 412 in fluid communication with a wound. Second elongated section 420 may be connected to a sealed, sterilized suction device (not shown) for drawing fluid through the bioabsorbable drain tube 400.

Bioabsorbable drain tube 400 is shown to be of octagonal configuration, although other tubular configurations will advantageously benefit from the novel features disclosed herein, as may be appreciated by those skilled in the art. As shown, first elongated section 412 includes a single lumen octagonal tube 426, with a plurality of drain openings 418 extending through the wall 428 of the single lumen octagonal tube 426, as shown in FIG. 10. The single lumen 436 permits fluid communication with the wound. The number and diameter of drain openings 418 may be varied in accordance with the intended application, so as to provide adequate tissue contact and drainage, while inhibiting tissue growth or entry of debris, such as clots, into the lumen 436.

Second elongated section 420 may also be molded from a bioabsorbable polymer of the type described hereinabove or, alternatively, may be molded from any of a number of other suitable materials, such as a biocompatible elastomer like silicone.

The second end of the first elongated section 412 and the first end of the second elongated section 420 may be connected together in abutting relationship by means of a flexible tubular collar (not shown). As may be appreciated, such a collar serves to span the butt joint formed by the second end 416 of first elongated section 412 and the first end 422 of second elongated section 420 and may be affixed to first elongated section 412 and second elongated section 420 by a suitable adhesive material.

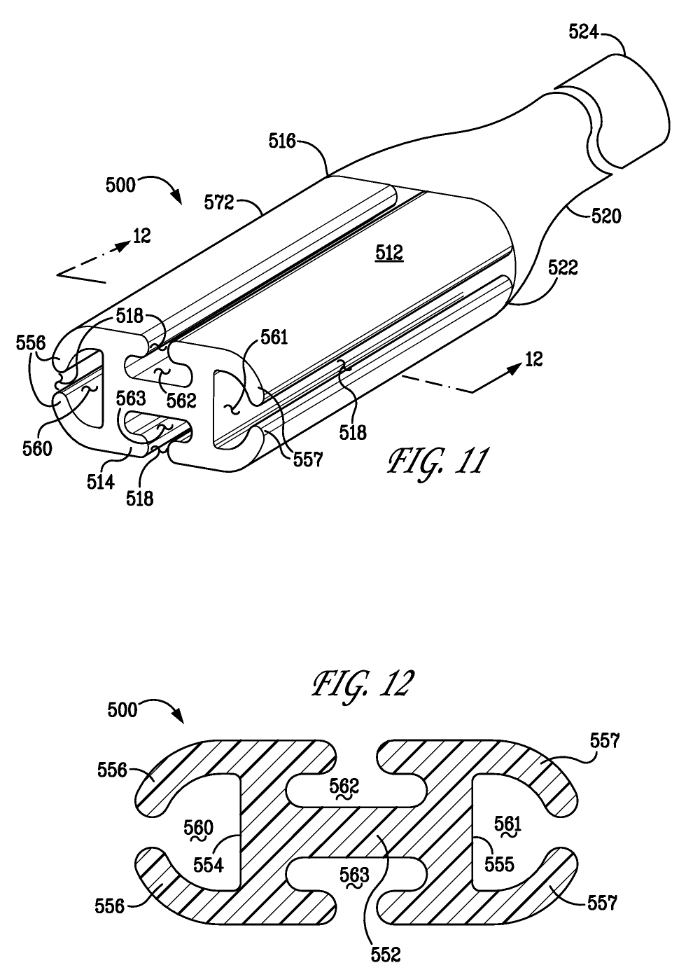

Referring now to FIGS. 11 and 12, a further embodiment of the present invention provides a bioabsorbable drain tube 500 having a generally oval cross-section. Bioabsorbable drain tube 500 includes a first elongated section 512 molded from a bioabsorbable polymer of a type described hereinabove. First elongated section 512 has a first end 514, a second end 516 and a plurality of drain openings 518. Bioabsorbable drain tube 500 also includes a second elongated section 520 having a first end 522 and a second end 524. As shown, the first end 522 of the second elongated section 520 is in fluid communication with the second end 516 of first elongated section 520.

As may be seen, drain tube 500 is configured so as to be substantially radially symmetrical. Of course, since the bioabsorbable drain tube 500 is oval, rather than round, the corresponding parts of the drain, referred to above with respect to radial symmetry will not be equidistant from the central axis. The bioabsorbable drain tube 500 also has diametrical symmetry, which, as used herein, means that for opposed radii (i.e., 180 degrees relative to each other), extending from a central axis, there are corresponding parts of the bioabsorbable drain tube on such radii, equidistant from the central axis, regardless of the orientation of such radii about the axis. While such symmetry has many of the same advantages as radial symmetry, the oval or flat configuration of the bioabsorbable drain tube 500 makes it particularly useful for draining areas between organs, or other areas where surgeons typically prefer drains having an oval or flat profile.

As with the other embodiments described hereinabove, first elongated section 512 and a small part of the second elongated section 520 are placed in a patients body with the first elongated section 512 in fluid communication with a wound or surgical site. According to one embodiment, second elongated section 520 is connected to a sealed, sterilized suction device (not shown) for drawing fluid through the bioabsorbable drain tube 500.

First elongated section 512 includes an axial core 552 perpendicularly connected at its ends to respective side rib portions 554 and 555. Respective outer peripheral members 556 are connected to each of the ends of the side rib portion 554. Similarly, respective outer peripheral members 557 are connected to each of the ends of the side rib portion 555. The ribs 554 and 555 and outer peripheral members 556 and 557 form plural T-shaped members radiating from the axial core 552. As best seen in FIG. 12, the two pairs of outer peripheral members 556 and 557, respectively, cooperate to form a segmented oval. As shown, the outer peripheral members 556 and 557 of the first elongated section 512 form longitudinal grooves 558 (see FIG. 11) throughout the length of the first elongated section 512. The pair of outer peripheral members 556 extends arcuately from the rib portion 554 and cooperates with the outer wall of the rib portion 554 to form a substantially semi-circular lumen 560. In like manner, the pair of outer peripheral members 557 extends arcuately from the rib portion 555 and cooperates with the outer wall of the rib portion 555 to form a second, essentially semi-circular, lumen 561, in opposed relationship to the lumen 560. Additionally, the outer peripheral members 556 and 557 extend on either side of the axial core 552, in parallel relationship thereto, to form a pair of essentially rectangular lumens 562 and 563 on opposite sides of the axial core 552. Thus, as shown in FIGS. 11 and 12, the first elongated section 512 has two side lumens 560 and 561, a top lumen 562, and a bottom lumen 563. Further, each of the lumens 560, 561, 562 and 563 has a respective longitudinal groove 558 for fluid communication with the wound. Therefore, drainage is provided from each of four sides of the first elongated section 512.

The second end of the first elongated section 512 and the first end of the second elongated section 520 may be connected together in abutting relationship by means of a flexible tubular collar (not shown). As may be appreciated, such a collar serves to span the buff joint formed by the second end 516 of first elongated section 512 and the first end 522 of second elongated section 520 and may be affixed to first elongated section 512 and second elongated section 520 by a suitable adhesive material.

Second elongated section 520 may also be molded from a bioabsorbable polymer of the type described hereinabove or, alternatively, may be molded from any of a number of other suitable materials, such as a biocompatible elastomer like silicone.

The drain tubes disclosed herein may be formed using any conventional molding or forming process. For example, the channel-type drain tubes may be advantageously formed in one step by any well-known extrusion processes.

As may be readily appreciated, the bioabsorbable drain tubes disclosed herein are designed to eliminate the need for removal from the patient upon completion of the drainage process, thus eliminating the pain normally associated with the removal procedure. Another benefit results from the fact that patients using the bioabsorbable drain tubes disclosed herein will not require the use of morphine, anti-inflammatory drugs, local anesthetics or inhalation agents as part of the analgesia for the drain tube removal procedure. Further benefits include reduced patient discomfort, wound irritation, and tissue damage. Moreover, this drain is safer and more reliable than comparable prior designs, and may advantageously be manufactured using relatively low cost, processes, such as by extrusion or other well known conventional processes. Thus, the drain of the present invention provides significant advances with respect to wound drain tubes for closed, deep wounds.

All patents, test procedures, and other documents cited herein, including priority documents, are fully incorporated by reference to the extent such disclosure is not inconsistent with this invention and for all jurisdictions in which such incorporation is permitted.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the invention, including all features which would be treated as equivalents thereof by those skilled in the art to which the invention pertains.

What is claimed is:

1. A bioabsorbable drain tube, comprising:
(a) a first elongated section molded from a bioabsorbable polymer, said first elongated section having a first end, a second end and a plurality of drain openings; and
(b) a second elongated section, said second elongated section having a first end and a second end, said first end of said second elongated section in fluid communication with said second end of said first elongated section.

2. The bioabsorbable drain tube of claim 1, wherein said bioabsorbable polymer comprises a biocompatible polymer chosen from poly(lactide), poly(glycolide), poly(dioxanone), poly(ε-caprolactone), poly(hydroxybutyrate) poly(β-hydroxybutyrate) poly(hydroxyvalerate), poly(tetramethyl carbonate), poly(lactide-co-glycolide), poly(amino acids) and copolymers and terpolymers thereof.

3. The bioabsorbable drain tube of claim 2, wherein said first elongated section further comprises an axial core having a longitudinal axis, said axial core having a plurality of radial ribs projecting radially along said longitudinal axis.

4. The bioabsorbable drain tube of claim 3, wherein said radial ribs are of equal length and have a first end and a second end, each second end terminating at said axial core's periphery and spaced equally about said axial core.

5. The bioabsorbable drain tube of claim 4, wherein each first end of each radial rib terminates in an outer peripheral member, said outer peripheral member extending longitudinally about the length of each radial rib.

6. The bioabsorbable drain tube of claim 5, wherein said outer peripheral members are sized to form a segmented circle or oval at said first elongated section's periphery with gaps between adjacent outer peripheral members.

7. The bioabsorbable drain tube of claim 6, wherein each gap forms a drain opening parallel to said longitudinal axis of said axial core and extending throughout the length of said first elongated section.

8. The bioabsorbable drain tube of claim 7, wherein said axial core, said radial ribs, said drain openings and said outer peripheral members form a plurality of lumens lengthwise along said first elongated section.

9. The bioabsorbable drain tube of claim 8, wherein said first elongated section includes three lumens.

10. The bioabsorbable drain tube of claim 8, wherein said first elongated section includes four lumens.

11. The bioabsorbable drain tube of claim 8, wherein each outer peripheral member is symmetrically positioned about each of said radial ribs.

12. The bioabsorbable drain tube of claim 8, wherein each outer peripheral member is asymmetrically positioned about each of said radial ribs.

13. The bioabsorbable drain tube of claim 2, wherein said first elongated section includes a single lumen tube having a plurality of drain openings extending therethrough.

14. The bioabsorbable drain tube of claim 13, wherein said single lumen tube is of circular cross-section.

15. The bioabsorbable drain tube of claim 13, wherein said single lumen tube is of octagonal cross-section.

16. The bioabsorbable drain tube of claim 1, wherein said second elongated section is molded from a bioabsorbable polymer.

17. The bioabsorbable drain tube of claim 16, wherein said bioabsorbable polymer comprises a biocompatible polymer chosen from poly(lactide), poly(glycolide), poly(dioxanone), poly(ε-caprolactone), poly(hydroxybutyrate) poly(β-hydroxybutyrate) poly(hydroxyvalerate), poly(tetramethyl carbonate), poly(lactide-co-glycolide), poly(amino acids) and copolymers and terpolymers thereof.

18. The bioabsorbable drain tube of claim 15 wherein said second elongated section is molded form a biocompatible elastomer.

19. The bioabsorbable drain tube of claim 15 wherein said biocompatible elastomer comprises silicone.

20. A method of draining fluid from a surgical site or wound of a patient the method comprising the steps of:
(a) inserting a bioabsorbable drain tube into the surgical site or wound of a patient;
(b) positioning the bioabsorbable drain tube within a region of fluid accumulation of the surgical site or wound;
(c) placing collection means in fluid communication with the bioabsorbable drain tube; and
(d) accumulating fluid in the collection means.
